(19) United States
(12) Patent Application Publication
(10) Pub. No.: US 2004/0117866 A1
(43) Pub. Date: Jun. 17, 2004

(54) Novel transgenic zebrafish, gene fragments and methods for producing transgenic zebrafish

(76) Inventor: Huai-Jen Tsai, Taipei (TW)

Correspondence Address: HARNESS, DICKEY & PIERCE, P.L.C., P.O.BOX 8910, RESTON, VA, 20195, US

(21) Appl. No.: 10/632,794

(22) Filed: Aug. 4, 2003

(30) Foreign Application Priority Data
Aug. 2, 2002 (TW) 091117396
Aug. 2, 2002 (TW) 091117397

(51) Int. Cl. A01K 67/027; C12N 15/861

(52) U.S. Cl. 800/20; 435/456; 435/320.1

(57) ABSTRACT

The invention relates to a recombinant DNA method for producing transgenic golden zebrafish (golden zebra danio). The invention also relates to novel gene fragments for producing the transgenic golden zebrafish. The invention further relates to novel transgenic golden zebrafish.

### FIELD OF THE INVENTION

The invention relates to a method for producing novel transgenic golden zebrafish (golden zebra danio). The invention also relates to novel gene fragments and novel transgenic golden zebrafish.

### BACKGROUND OF THE INVENTION

Ornamental fish is one sector of the fishery business and belongs to entertainment industry with global business. Therefore, the use of recombinant DNA and transgenic techniques into the modification of ornamental fish could make a good economic effect.

Unfortunately, the conventional transgenic technology can only produce transgenic fish with the expression of emitting mosaic or weak fluorescence. Such fish should be found under fluorescent microscope with a specific wavelength. Due to the impracticality and various difficulties, the previous fluorescent fish species were not well-received by the consumers and do not have commercial value for ornamental fish.

### SUMMARY OF THE INVENTION

The object of the invention is to apply recombinant DNA techniques on commercially available plasmid constructs such as pDsRed2-1 from Clontech and p-αEGFPITR, to establish an stable supply of seedlings with desired transgenes.

Another object of the invention relates to a gene fragment comprising (1) α-actin gene promoter of golden zebrafish; (2) fluorescence gene; (3) inverted terminal repeats of adeno-associated virus; and (4) a basic part from pUC, which results in a new species of golden zebrafish whose skeletal muscle emits red fluorescence.

Yet another object of the invention relates to the method of engineering a novel golden zebrafish which carry the fluorescent transgene and express fluorescent protein in their systemic skeletal muscle.

### BRIEF DESCRIPTION OF THE DRAWINGS

FIG. 1 illustrates the plasmid construct, p-αEGFPITR, with its restriction sites;

FIG. 2 illustrates the plasmid construct, pDsRedITR, with its restriction sites.

FIG. 3 illustrates the plasmid construct, p-αDsRedITR, with the inserted gene fragment and restriction sites.

FIG. 4 is a linear graphic depiction of the DNA fragment p-αDsRedITR and restriction sites.

FIG. 5 is a linear graphic depiction of the DNA fragment p-αEGFPITR and restriction sites.

FIG. 6 is a photographic representation of a golden zebrafish embryo (F1 generation) successfully transfected with DNA of the invention after three days, demonstrating the expression of red fluorescence. Photographic is exposed at 1/4 second.

FIG. 7 is a photographic representation of a golden zebrafish embryo (F1 generation) successfully transfected with DNA of the invention after three days, demonstrating the expression of green fluorescence. Photographic is exposed at 2 seconds.

FIG. 8 illustrates the inheritance/expression rates of the novel golden zebrafish (with p-αDsRedITR transgene) at different generations (F0, F1, and F2).

### DETAILED DESCRIPTION OF THE INVENTION

To avoid the disadvantages of the prior art, the current invention is of thorough and stringent design, and with conceptual breakthrough. That is, the α-actin gene promoter of golden zebrafish is introduced into such as pDsRedITR, to get a novel plasmid construct, p-αDsRedITR. Then, p-αDsRedITR, will be micro-injected into the cytoplasm of fertilized eggs (prior to first cleavage) of golden zebrafish. These eggs will be used to screen for progeny with fluorescent transgene that will be expressed throughout the fish on their systemic skeletal muscle. Progeny with fluorescent transgene will be used for future breeding.

The method of the invention can provide the following five improvements over other available methods:

1. The main material constructs are plasmid constructs p-αEGFPITR and such as pDsRedITR, which are of stable and economical sources.

2. Novel DNA fragments will enable the golden zebrafish systemic skeletal muscle fluorescence.

3. The microinjected of novel DNA fragments into fertilized eggs, will enable the golden zebrafish systemic skeletal muscle fluorescence at a higher ratio, and with better quality.

4. The heterologous transgene will be stably passed down to the next generation. This will enable economical and natural method of reproduction.

5. In novel mutated golden zebrafish species, its systemic skeletal muscle will emit a fluorescent light, easily seen by naked eyes. Under light source of shorter wavelength, the red fluorescence of the fish will be intensified to bring out its special characteristics and beauty. This will be an extra value-add to ornamental fish.

The fluorescent gene used in the invention can be red fluorescent gene such as DsRed, which can be obtained from pDsRed2-1 of Clontech, or can be green fluorescent gene such as GFP, which can be purchased from Amersham Bioscience.

Given the above, the present invention provide a method of producing golden zebrafish with systemic fluorescence comprising:

(a) constructing a plasmid including ITR, CMV promotor, fluorescent gene, S40 poly A and ITR from upstream to downstream;

(b) replacing the CMV promotor with α-actin gene promoter (systemic skeletal muscle actin gene expression) of golden zebrafish to produce a new plasmid construct;

(c) linearizing the new plasmid construct;

(d) microinjecting the linearized plasmid construct into fertilized eggs of golden zebrafish;

(e) selecting the eggs with fluorescence; and

(f) cultivating the eggs to produce golden zebrafish with systemic fluorescence.

The linearized plasmid is preferred to select from

[image]

or

[image]

The preferred fluorescent gene used in the method of the invention is red fluorescent gene from pDsRed2-1 or green fluorescent gene from pEGFP-1.

The present invention also provides golden zebrafish with systemic fluorescence produced from the method of the invention. The preferred golden zebrafish have systemic red or green fluorescence.

### EXAMPLES

The examples below are non-limiting and are merely representative of various aspects and features of the present invention.

The method for producing golden zebrafish with red fluorescence

1. A commercially available plasmid construct, pDsRed2-1 and p-αEGFPITR were selected as the basic materials. The plasmid construct pDsRed2-1 could be purchased from Clontech. The plasmid construct p-αEGFPITR could be produced according to the description of the related literature such as “Uniform GFP-expression in transgenic medaka (Oryzias latipes) at the F0 generation,” Chi-Yuan Chou et al., Transgenic Research 10: 303-315, 2001.

2. The DsRed fragment was spliced out from plasmd pDsRED2-1. Then, CMV promotor and two adeno-associated virus inverted terminal repeats (ITR) were linked with the DsRed fragment as depicted in FIG. 2 to produce plasmid construct p-DsRedITR. The plasmid construct p-DsRedITR achieved a greater degree of expression stability.

3. Formation of the Novel Plasmid Construct: p-αDsRedITR

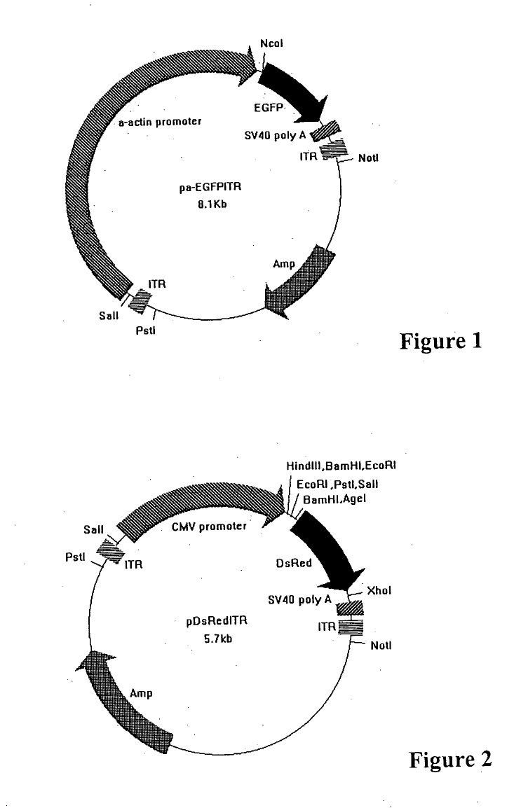

As illustrated in FIG. 1, the golden zebrafish α-actin gene promoter was obtained from the plasmid construct p-αEGFPITR by way of digesting with restriction enzymes Nco I and Sal I. Nco I was first used to digest the plasmid construct, ends were filled in, followed by a subsequent digestion with Sal I to obtain a 3.68 kb fragment.

As illustrated in FIG. 2, the CMV promoter was spliced out from the plasmid construct, pDsRedITR by way of digesting with restriction enzymes Bam HI and Sal I. Bam HI was first used to digest the plasmid construct, ends were filled in, followed by a subsequent digestion with Sal I to obtain a 4.2 kb fragment. Then, the α-actin gene promoter (systemic skeletal muscle actin gene expression) of golden zebrafish was ligated onto the plasmid construct, pDsRedITR at the position where the CMV promoter was spliced out from. The resulting plasmid construct had two 145 bp of adeno-associated virus inverted terminal repeats (ITR). One ITR was located at 3′ end of SV40 poly A. The other was located at 5′ end of α-actin gene promoter.

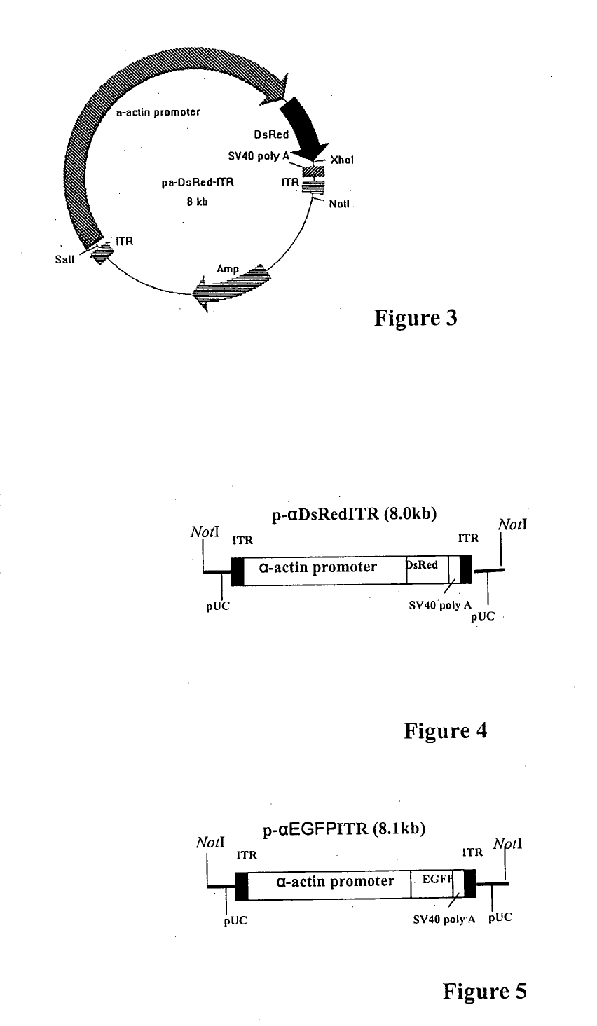

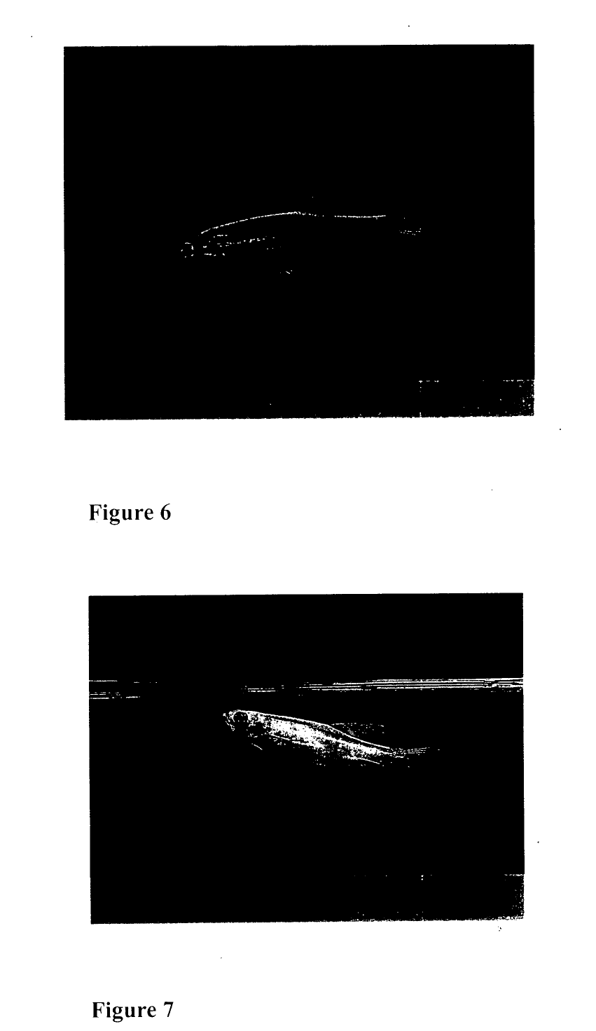

As illustrated in FIG. 3, the resulting plasmid construct, p-αDsRedITR, with a total length of 8.0 kb was made. The plasmid construct α-αDsRedITR contained (1) golden zebrafish α-actin gene promoter (systemic gene expression); (2) sea coral red fluorescent protein; (3) adeno-associated virus inverted terminal repeats; and (4) pUC plasmid construct basis.

The plasmid construct p-αDsRedITR was introduced into Escherichia coli 5α to be produced asexually in great quantity.

4. Linearization of Plasmid Construct:

As illustrated in FIG. 4, suitable amount of DNA from p-αDsRedITR was digested with proportional amount of Not I restriction enzyme, and a small amount of digested product was analyzed by agarose gel electrophoresis, to verify its linearity and the desired fragment length is in fact 8 kb. Then, the digested DNA products were extracted by a solution of equal volume of phenol:chloroform (1:1), precipitated by ethanol, air dried, then dissolved in PBS at a density of 10 μg/ml, which will be used for cytoplasmic micro-injection.

5. Cytoplasmic Micro-Injection

a. Collection of fertilized eggs: At 11 pm of the night before microinjection, and before the incubator entered dark cycle, fish were collected in a boxed area and were separated by separation net. On the next morning and after the light cycle begins, fish eggs were collected every 15-20 minutes. Every microinjection session, 30-40 eggs were injected; and during every experiment, 250-300 eggs were injected.

b. Microinjection: Linearized DNA was quantified and dissolved in 5×PBS with diluted phenol red to the desired concentration. DNA was picked up by micro-capillary of zebrafish microinjector (Drummond) wherein injection needle width of the micro-capillary was approximately 10 μm. As micro-needle enters cell cytoplasm, DNA injected was approximated to be about 2,3 nl.

c. Cultivation of fertilized eggs: Injected eggs were rinsed with sterilized solution, cultured in incubator wherein the temperature was defined at 28.5° C. The fluorescence could be observed in developing embryo after 24 hours.

6. Fluorescent Microscopy Observation:

The injected embryo was placed in dish containing water. The distribution and performance of red fluorescence could be observed under fluorescence microscope (Leica MZ-12; Fluorescence System: light source Hg 100 W; main emission wavelength 558 nm, and main absorption wavelength 583 nm, filter set RFP-Plus; photography system MPS60).

7. Germ-Line Transmission of Transgene:

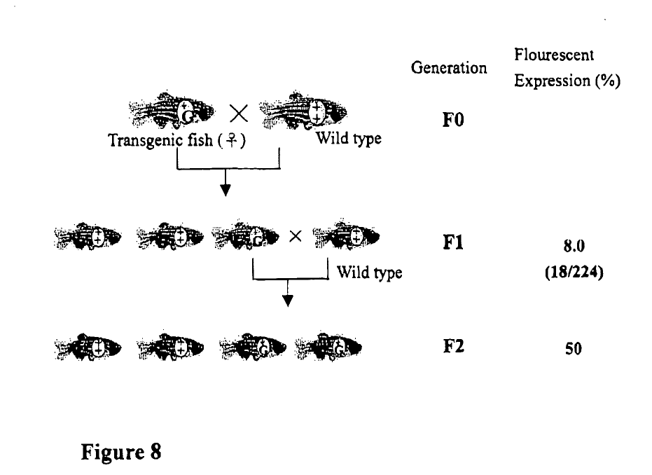

As showed in FIG. 8, mutated novel golden zebrafish (F0) originated from embryos of microinjection with p-αDsRedITR fragment and with systemic skeletal muscle expression of red fluorescence protein, were crossed with wild type, to get F1 progeny that exhibited uniform fluorescence. Then, the F1 with fluorescence expression was again crossed with wild type to get F2 progeny, which all exhibited red fluorescent expression, and can be readily distinguished from fish without fluorescent expression. The difference between transgenic golden zebrafish and wild type could be even better discerned under blue light. The DNA fragment of the invention could be modified to carry other fluorescent genes, and thus fish with different fluorescence could be produced.

Other fluorescent transgene may be introduced into golden zebrafish eggs with red fluorescence to make fish with various body colors.

The golden zebrafish of the invention can be broadly applied to medicines and researches in various life sciences, for example, cell fusions, cloning, nuclear transfer, cell motility, cell targeting, and embryonic development research.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The embryos, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

What is claimed is:

1. A gene fragment comprising (1) α-actin gene promoter of golden zebrafish; (2) fluorescence gene; (3) inverted terminal repeats (ITR) of adeno-associated virus; and (4) a basic part from pUC.

2. The fragment of claim 1 which is FIG. 4 of the drawings or FIG. 5 of the drawings.

3. A method of producing golden zebrafish with systemic fluorescence comprising:
(a) constructing a plasmid including ITR, CMV promotor, fluorescent gene, S40 poly A and ITR from upstream to downstream;
(b) replacing the CMV promotor with α-actin gene promoter (systemic skeletal muscle actin gene expression) of golden zebrafish to produce a new plasmid construct;
(c) linearizing the new plasmid construct;
(d) microinjecting the linearized plasmid construct into fertilized eggs of golden zebrafish;
(e) selecting the eggs with fluorescence; and
(f) cultivating the eggs to produce golden zebrafish with systemic fluorescence.

4. The method of claim 3 wherein the linearized plasmid is FIG. 4 of the drawings or FIG. 5 of the drawings.

5. The method of claim 3 wherein the fluorescent gene is red fluorescent gene from pDsRed2-1.

6. The method of claim 3 wherein the fluorescent gene is green fluorescent gene from pEGFP-1.

7. A golden zebrafish with systemic fluorescence produced from the method of claim 3.

8. The golden zebrafish of claim 7 which has systemic red fluorescence.

9. The golden zebrafish of claim 7 which has systemic green fluorescence.
